(19) United States
(12) Patent Application Publication
(10) Pub. No.: US 2023/0172468 A1
(43) Pub. Date: Jun. 8, 2023

(54) PPG AND ECG SENSORS FOR SMART GLASSES

(71) Applicant: Meta Platforms Technologies, LLC, Menlo Park, CA (US)

(72) Inventors: Kirsten Kaplan, Redmond, WA (US); Robert Bruce Darling, Lake Forest Park, WA (US); Rex Crossen, Oakland, CA (US); Hind Hobeika, San Francisco, CA (US); Sebastian Sztuk, Virum (DK); Johana Gabriela Coyoc Escudero, Union City, CA (US); Nan Wang, Sunnyvale, CA (US)

(21) Appl. No.: 18/060,491

(22) Filed: Nov. 30, 2022

Related U.S. Application Data
(60) Provisional application No. 63/285,907, filed on Dec. 3, 2021.
(60) Provisional application No. 63/285,913, filed on Dec. 3, 2021.
(60) Provisional application No. 63/388,168, filed on Jul. 11, 2022.

(51) Int. Cl. A61B 5/024; A61B 5/0245; A61B 5/291; A61B 5/322; A61B 5/00

(52) U.S. Cl. CPC A61B 5/02438; A61B 5/02427; A61B 5/0245; A61B 5/291; A61B 5/322; A61B 5/7235; A61B 5/7282; A61B 5/6803

(57) ABSTRACT

A smart glass including photoplethysmography and electrocardiogram sensors to determine a health condition of the user is provided. The smart glass includes a frame for holding two eyepieces, the frame having two nose pads to rest on a user's nose, and two arms to rest on two user's ears, a sensor mounted on at least one of the nose pads or the arms, and configured to collect an optical signal from a user's blood vessel, and a processor configured to obtain a waveform from the optical signal or the electrical signal, and to determine a cardiovascular parameter based on the waveform.

The present disclosure is related and claims priority under 35 U.S.C. § 119(e) to U.S. Prov. Appln. No. 63/285,913, entitled ADAPTIVE SENSORS TO ASSESS USER CONDITION FOR WEARABLE DEVICES to Doruk SENKAL et al., filed on Dec. 3, 2021, to U.S. Prov. Appln. No. 63/285,907, entitled ADAPTIVE USER INTERFACES FOR WEARABLE DEVICES, TO Nan WANG et al., filed on Dec. 3, 2021, and to U.S. Prov. Appln. No. 63/388,168, entitled PPG AND ECG SENSORS FOR SMART GLASSES, to Kirsten KAPLAN, et al., filed on Jul. 11, 2022, the contents of which applications are hereinafter incorporated by reference in their entirety for all purposes.

### BACKGROUND

### Background

### Field

The present disclosure is directed to optical and electrical sensors in augmented reality (AR) headsets (e.g., smart glasses). More specifically, embodiments as disclosed herein are directed to photoplethysmography (PPG), electrocardiogram (ECG), and electro-encephalogram (EEG) sensors to determine a health condition for smart glass users.

### Related Art

In the field of wearable devices, many applications include collecting data from sensors mounted on the wearable devices that enable assessment of different conditions of a user. However, many head-based wearable devices lack the capability of measuring signal from the brain, ear, eyes, and other important organs in the vicinity of the head, thereby leaving untapped a wealth of critical health information.

### SUMMARY

In a first embodiment, a headset includes a frame for holding two eyepieces, the frame having two nose pads to rest on a user's nose, and two arms to rest on two user's ears, a sensor mounted on at least one of the nose pads or the arms, and configured to collect an optical signal from a user's perfused tissue, and a processor configured to obtain a waveform from the optical signal and to determine a cardiovascular parameter based on the waveform.

In a second embodiment, a computer-implemented method includes directing a first optical signal to a first point in a perfused tissue of a subject, collecting, from the first point in the perfused tissue of the subject, an interacted portion of the first optical signal, forming a first waveform with the interacted portion of the first optical signal, and determining a cardiovascular value of the subject based on the first waveform.

In a third embodiment, a computer-implemented method includes collecting, from a subject's skin, a first electrical signal, forming a first waveform with the first electrical signal, identifying, based on the first waveform, one of a cardiovascular activity of a subject or an encephalographic activity of the subject, and determining a health parameter of the subject based on the first waveform.

In other embodiments, a non-transitory, computer-readable medium stores instructions which, when executed by a processor, cause a computer to perform a method, the method includes directing a first optical signal to a first point in a perfused tissue of a subject, collecting, from the first point of the perfused tissue of the subject, an interacted portion of the first optical signal, forming a first waveform with the interacted portion of the first optical signal, and determining a cardiovascular value of the subject based on the first waveform.

In yet other embodiments, a system includes a first means to store instructions and a second means to execute the instructions to cause the system to perform a method. The method includes directing a first optical signal to a first point in a perfused tissue of a subject, collecting, from the first point in the perfused tissue of the subject, an interacted portion of the first optical signal, forming a first waveform with the interacted portion of the first optical signal, and determining a cardiovascular value of the subject based on the first waveform.

These and other embodiments will become clear to one of ordinary skill in the art in view of the following.

### BRIEF DESCRIPTION OF THE FIGURES

FIG. 1 illustrates an architecture including one or more wearable devices coupled to one another, to a mobile device, a remote server and to a database, according to some embodiments.

FIG. 2 illustrates different locations for PPG sensors in a smart glass, according to some embodiments.

FIG. 3 illustrates a configuration for PPG sensors in an arm of a smart glass, according to some embodiments.

FIGS. 4A-4C illustrate the wiring and signaling configuration for PPG sensors in nose pads of a smart glass or headset, according to some embodiments.

FIG. 5 illustrates waveforms in different configurations in a PPG sensor mounted on a smart glass or headset, according to some embodiments

FIG. 6 illustrates different locations for electrodes in an ECG or EEG sensors in a smart glass or headset, according to some embodiments.

FIG. 7 is a flowchart illustrating steps in a method for determining a cardiovascular condition of an AR headset user with a PPG sensor, an ECG sensor, an EEG sensor, an EOG sensor or any combination thereof, according to some embodiments.

FIG. 8 is a flowchart illustrating steps in a method 800 for determining a cardiovascular condition of an AR headset user with an ECG and an EEG sensor, according to some embodiments.

FIG. 9 is a block diagram illustrating details and components of a computer system for use in the architecture of FIG. 1 and to perform the method of FIGS. 7 and 8, according to some embodiments.

In the figures, elements having the same or similar reference numeral are associated with the same or similar attributes and features, unless explicitly stated otherwise.

### DETAILED DESCRIPTION

In the following detailed description, numerous specific details are set forth to provide a full understanding of the present disclosure. It will be apparent, however, to one ordinarily skilled in the art, that embodiments of the present disclosure may be practiced without some of these specific details. In other instances, well-known structures and techniques have not been shown in detail so as not to obscure the disclosure.

### General Overview

Wearable devices for the user's head offer the convenience that the head has more damped motion, with less interferences or noise (motion of the head is more often than not intentional, measured, and conscious). On the other hand, there are challenges to ensure, for example, that smart glasses do not slip during motion (glasses may be less securely attached than wrist-bands or a wrist-watch). This slippage is common in devices that include heavier electronic components on the glasses and results in user discomfort as it requires constant fixing of the frame and/or even holding the glasses with one hand while in operation.

In some embodiments, smart glasses include photoplethysmography (PPG) sensors configured to optically measure blood vessels and capillaries to provide a heart rate monitoring, pulse oximetry readings, and the like. In some embodiments, an ear placement (e.g., on the arms of a headset or smart glass) may include an array of PPG sensors to allow selection of an active sensor on a per-user basis. This ensures optimal signal quality.

A PPG sensor as disclosed herein may include an emitter of electromagnetic radiation and a receiver of the electromagnetic radiation reflected off (e.g., reflective mode), or transmitted through (e.g., transmissive mode), perfused tissue including one or more blood vessels. PPG sensors as disclosed herein may be placed on a single nose pad or on the arms of a smart glass or headset, close to the user's nose or ears, operating in reflective mode. PPG sensors having an emitter and receiver on opposite nose pads in the smart glass may operate in a transmissive mode. Variations in the electromagnetic radiation scattered off of the blood vessel due to the pulsation of the blood vessel as the heart cycles through, follow quite accurately the heart pulse. The electromagnetic radiation may include light generated by a laser or a light emitting diode (LED) in the visible wavelength range (e.g., blue, green, or red), in the near-infrared wavelength range (NIR, e.g., between 750-2500 nm), or in the infrared wavelength range (IR, e.g., between 2500-10000 nm or more). Other wavelength and frequency ranges for PPG sensors as disclosed herein may be selected, without departing from the scope of the present disclosure. For transmissive mode a red, IR, or NIR emitter may be desirable due to the longer penetration depth through human skin. For reflective mode, a green LED may be desirable due to the higher scattering intensity at lower wavelengths, providing a larger reflective signal on the receiver (e.g., photodetector).

In some embodiments, PPG sensors may include a green LED for heart rate (HR) measurements. Other LED colors for the emitter in PPG sensors as disclosed herein may be chosen for blood oxygenation measurements (e.g., Red/IR), and potentially Blue/Green/Red/IR for multi-wavelength blood pressure measurements. In some embodiments, users may prefer PPG sensors with IR LED emitters to avoid distraction/annoyance by onlookers and people in the surroundings of the user. To avoid the above issue, some embodiments may include a pulsating or other time interleaving scheme to reduces the external visibility of the emitter signals in PPG sensors as disclosed herein.

PPG sensors as disclosed herein may provide waveforms indicative of various cardiovascular-related diseases such as atherosclerosis and other conditions associated with varying degrees of arterial stiffness. Furthermore, the time derivatives of a PPG sensor waveform may also be indicative of cardiovascular illnesses that may affect the user in the future.

Embodiments as disclosed herein include optical and electrical wearable sensors that may be combined to enable passive, continuous monitoring of blood pressure, electro-cardiogram (ECG) signals, and electro-encephalogram (EEG) signals around the head area (e.g., nose and ears) detected by smart glass wearable devices. Measuring ECG signals across the head enables cardiovascular assessments without requesting active effort on the part of the user (e.g., as usual in wrist-based technologies). In some embodiments, ECG and PPG signals can be used to measure pulse transit time (e.g., comparing a PPG signal from the wrist with a PPG signal from the head), or pulse arrival time (e.g., comparing a PPG signal from either the head/wrist with an ECG signal), which can be correlated with blood pressure.

Optical and electrical wearable sensors as disclosed herein may be combined to enable passive, continuous monitoring of blood pressure (BP). For example, ECG and PPG signals can be combined and compared to measure pulse transit time (PTT, e.g., between wrist and head) which may be correlated with BP. The ECG collected across the head enables BP measurement without active effort on the part of the user, unlike wrist-based technologies. PPG and ECG sensors as disclosed herein operate through the skin, do not break the skin barrier and are thus non-invasive in that sense.

Example System Architecture

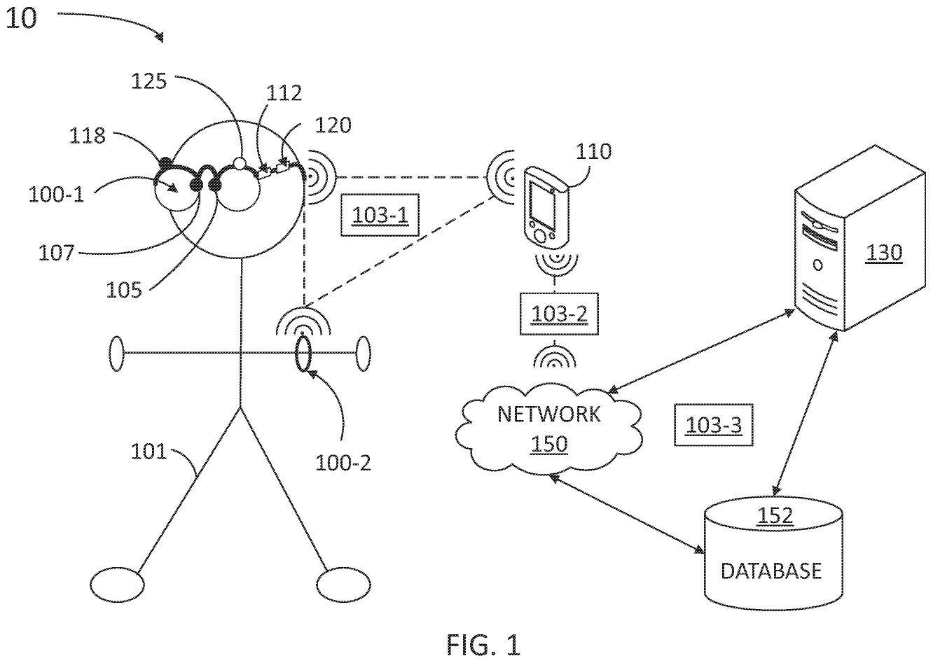

FIG. 1 illustrates an architecture 10 including one or more wearable devices 100-1 and 100-2 (hereinafter, collectively referred to as “wearable devices 100”) coupled to one another, to a mobile device 110, a remote server 130 and to a database 152, according to some embodiments. Wearable devices 100 may include a smart glass or headset 100-1 and a wrist-band (or “watch”) 100-2, and mobile device 110 may be a smart phone, all of which may communicate with one another via a communications module 118 and exchange a first dataset 103-1. Dataset 103-1 may include a recorded video, audio, or some other file or streaming media. A user 101 of wearable devices 100 may also be the owner or is associated with mobile device 110. In some embodiments, at least one of wearable devices 100 (e.g., the smart glass) may directly communicate with remote server 130, database 152, or any other client device (e.g., a smart phone of a different user, and the like) via a network 150.

Communications module 118 may be configured to interface with a network 150 to send and receive information, such as dataset 103-1, dataset 103-2, and dataset 103-3, requests, responses, and commands to other devices on network 150. In some embodiments, communications module 118 can include, for example, modems or Ethernet cards. Client devices 110 may in turn be communicatively coupled with a remote server 130 and a database 152, through network 150, and transmit/share information, files, and the like with one another (e.g., dataset 103-2 and dataset 103-3). Datasets 103-1, 103-2, and 103-3 will be collectively referred to, hereinafter, as “datasets 103.” Network 150 may include, for example, any one or more of a local area network (LAN), a wide area network (WAN), body-area-networks (BAN), the Internet, and the like. Further, the network can include, but is not limited to, any one or more of the following network topologies, including a bus network, a star network, a ring network, a mesh network, a star-bus network, tree or hierarchical network, and the like.

Headset 100-1 may include a PPG sensor 107 and an ECG or an EEG sensor 105, according to some embodiments. ECG/EEG sensor 105 may include one or more electrodes configured to detect electric signals such as currents, voltages, and potential changes generated by the nervous system or muscular tissue in the face or head of user 101. In some embodiments, smart glass or headset 100-1 may include one or more sensors 125 such as inertial measurement units (IMUs), gyroscopes, microphones, cameras, and the like mounted within the frame of the headset or wrist-watch or wrist-band. Other sensors 125 may include magnetometers, microphones, photodiodes and cameras, touch sensors, and other electromagnetic devices such as capacitive sensors, a pressure sensor, and the like. The microphones may include a contact microphone and an acoustic microphone.

In addition, headset 100-1, and any other wearable device 100, or mobile device 110 may include a memory circuit 120 storing instructions, and a processor circuit 112 configured to execute the instructions to cause headset 100-1 to perform, at least partially, some of the steps in methods consistent with the present disclosure.

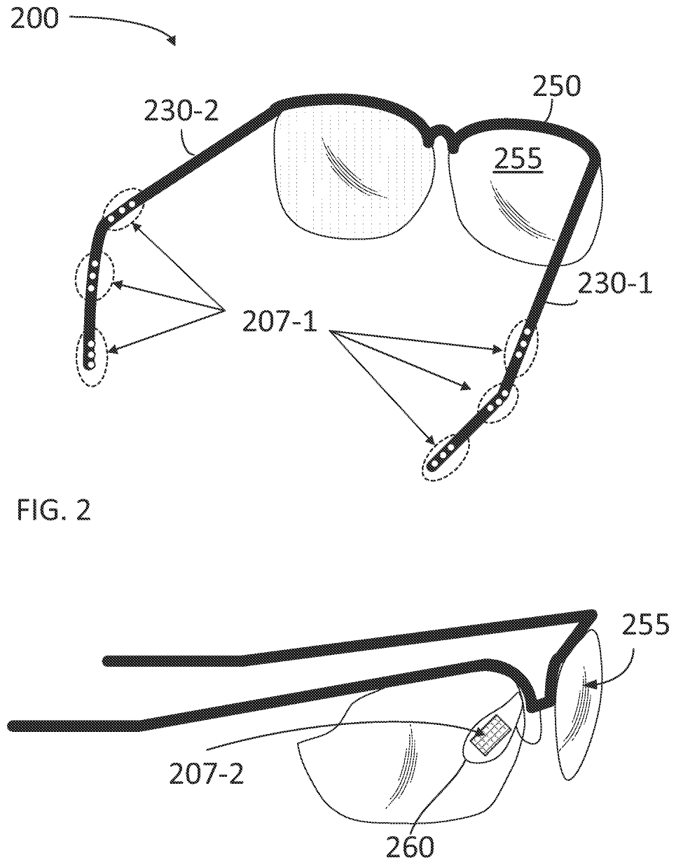

FIG. 2 illustrates different locations for PPG sensors 207-1 and 207-2 (hereinafter, collectively referred to as “PPG sensors 207”) in a smart glass 200, according to some embodiments. Smart glass 200 is a headset including a frame 250 holding two eyepieces 255 and having two arms 230-1 and 230-2 (hereinafter, collectively referred to as “arms 230”). One of the locations for sensors 207 includes one or both of the arms 230 of smart glass 200, close to the user's ear. Another location is on nose pads 260 of smart glass 200, close to the user's nose, with sensors disposed on the nose pad of the smart glasses.

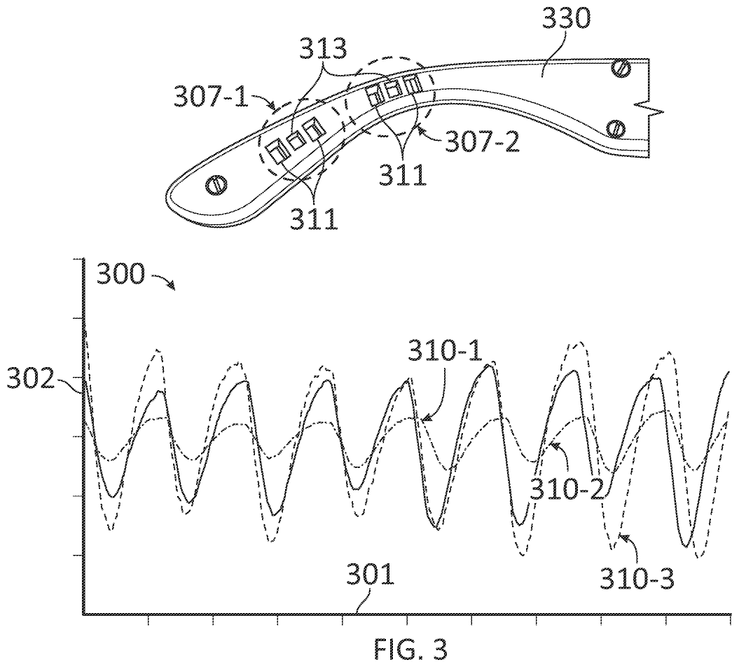

FIG. 3 illustrates a configuration for PPG sensors 307-1 and 307-2 (hereinafter, collectively referred to as “PPG sensors 307”) in an arm 330 of a smart glass, according to some embodiments. The configuration includes two sensing locations, each having an emitter (LED) 313 in the middle (e.g., a green LED to operate in reflective mode), surrounded by two photodetectors 311 (PD) on either side of emitter 313. In some embodiments, multiple photodetectors 311 per emitter 313 (e.g., three, four, or even more, as dimensions allow) may be included to ensure a stronger signal.

In some embodiments, a configuration may include two, three, or even more measurement sites to guarantee that a good signal be obtained at least from one site. The exact location of measurement sites along arm 330 may be chosen for optimal coverage (e.g., close to the occipital/temporal region around the user's ear) across a diverse user population.

A chart 300 having a temporal abscissa 301 (e.g., in seconds) and a PPG signal amplitude 302 ordinate, illustrates PPG waveforms 310-1 (emitter 313 operating at 10 milliamp—mA—), 310-2 (emitter 313 operating at 25 mA), and 310-3 (emitter 313 operating at 50 mA). Hereinafter, PPG waveforms 310-1, 310-2, and 310-3 will be collectively referred to as “PPG waveforms 310.” As can be seen from PPG waveforms 310, the signal and signal-to-noise ratio from PPG sensors 307 increases with increased power in emitter 313. This may be counterbalanced with the desire from users to make sensing devices look less conspicuous (especially for green LEDs).

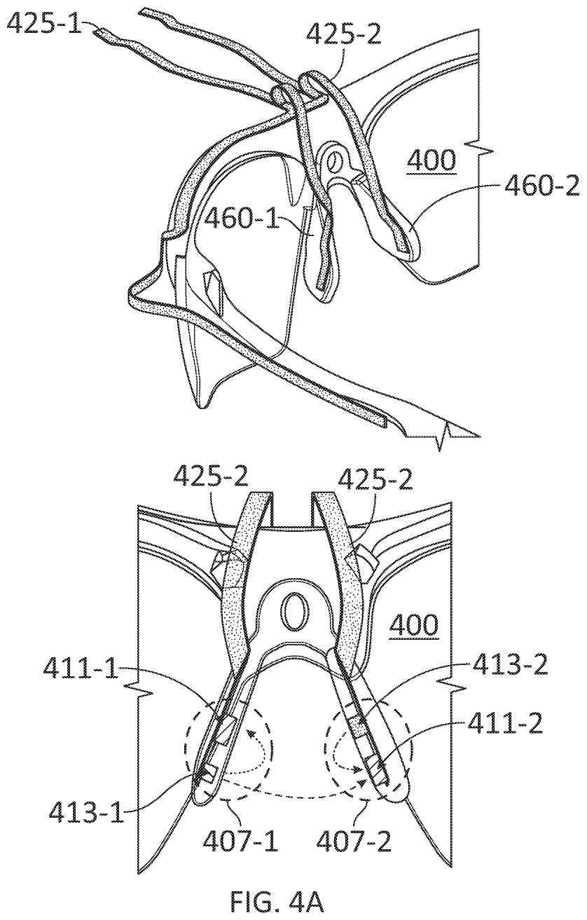

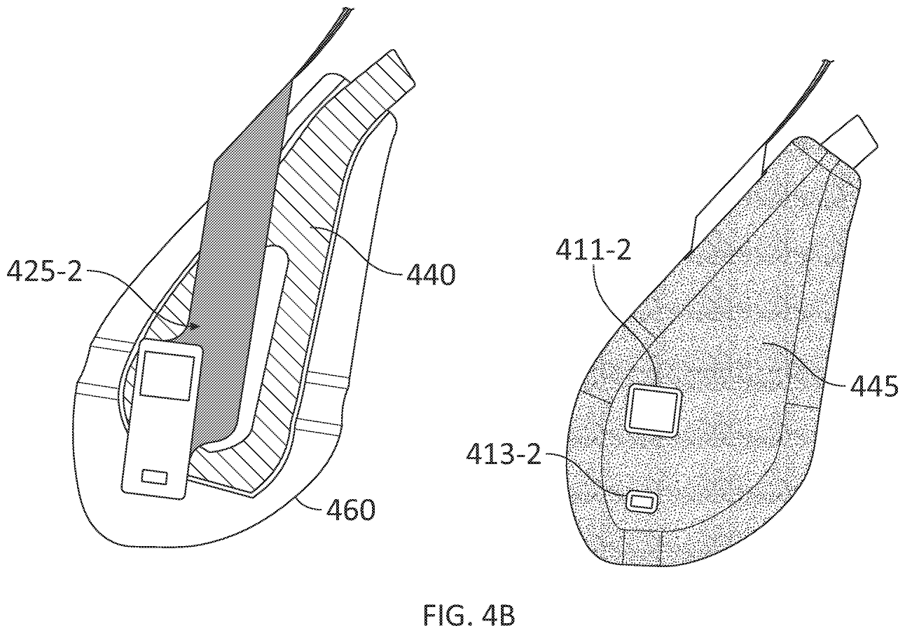

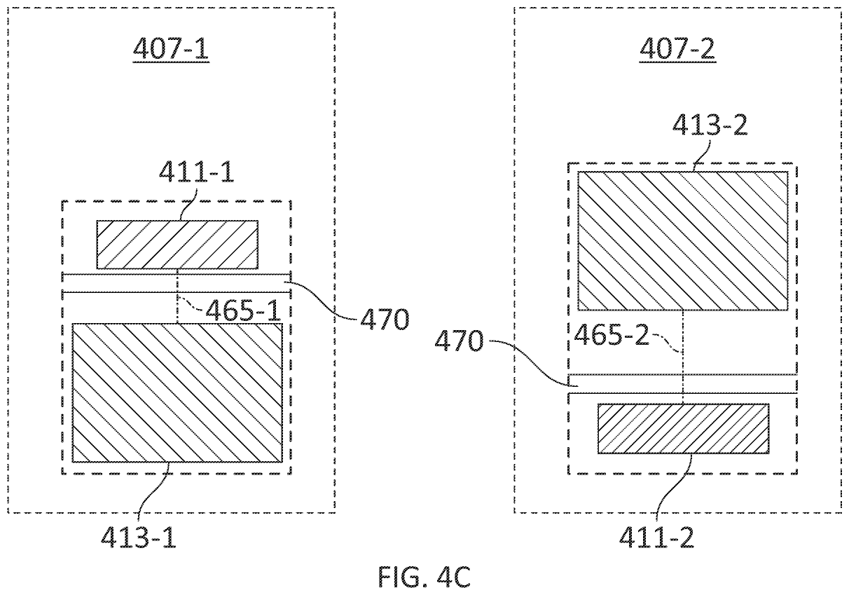

FIGS. 4A-4C illustrate the wiring and signaling configuration for PPG sensors 407 in nose pads 460-1 and 460-2 (hereinafter, collectively referred to as “nose pads 460”) of a smart glass or headset 400, according to some embodiments. In some embodiments, nose pads 460 may include bendable of flexible support bracket 440 (e.g., for sports class smart glasses). Some embodiments may include materials that do not provide full, long-term flexibility in the nose (for strong support of smart glass 400). Nose pads 460 may be custom designed to achieve better contact/fit across a population. Additionally, smart glass 400 may include highly adaptable designs and materials that provide a good fit across a diverse population, such as casing 445 in different colors, opaque, or even transparent. This is important to guarantee that at least one of PPG sensors 407 will provide a useful signal for most or all users. One of the conveniences of using nose pads 460 to mount PPG sensors 407 is the ability to use sheet metal pieces (e.g., bracket 440) to shape nose pads 460 with an angle in the coronal plane and in the transverse plane so as to snugly fit on the user's nose. This guarantees proper contact between emitters 413-1 and 413-2 (hereinafter, collectively referred to as “emitters 413”) and receivers 411-1 and 411-2 (hereinafter, collectively referred to as “receivers 411”) with the user's skin, enhancing signal-to-noise (SNR) ratio.

Note that IR LED emitter 413-1 is aligned with receiver 411-2 to maximize the signal through the user's nose in transmission mode. In some embodiments, PPG sensors 407 may operate in reflection mode, separately. In yet other embodiments, one or more waveforms may be collected simultaneously, or multiplexed in time, from each of receivers 411 in the first and second nose pads 460. Electrical connectors 425-1 and 425-2 (hereinafter, collectively referred to as “connectors 425”) provide power and control signals to PPG sensors 407. In some embodiments, each of connectors 425 may include multiple wires in a flexible ribbon, insulated from one another and other metal parts and connectors.

FIG. 4A illustrates a measurement configuration wherein an IR emitter 413-1 and receiver 411-1 pair is used in reflective mode (PPG 407-1), or in transmissive mode (emitter 413-1 and receiver 411-2 from either one of PPG sensors 407). A green emitter 413-2, coupled with receiver 411-2 is used in a reflective configuration (PPG 407-2). Electrical connectors 425-1 and 425-2 (hereinafter, collectively referred to as “connectors 425”), provide power an retrieve signals from PPG sensors 407.

FIG. 4B illustrates some of the interconnects used in the nose pad for powering and receiving signals from PPG sensors installed therein.

FIG. 4C illustrates an optical barrier between LED and PD (e.g., more than about 0.8 mm), to prevent crosstalk. The configuration of the PPG sensor illustrates a green LED emitter/receiver pair in a first nose pad and an IR LED emitter/receiver pair in the second nose pad. A spacing 465-1 between emitter 413-2 and receiver 411-2 in the green emitter/receiver configuration (about 1.5 mm, in some embodiments) is typically less than a spacing 465-2 between IR emitter 413-1 and receiver 411-2 (about 3 mm, in some embodiments) due to the higher scattering of green light. PPG sensor 407-2 is larger than PPG sensor 407-1 because IR light penetrates deeper into skin and bone tissue, while green light has larger proportional losses at any given distance, due to scattering. A light barrier 470 separating emitters 413 from sensors 407 may prevent any light not coming from the user's skin to activate or generate a background or interference signal in PPG sensors 407.

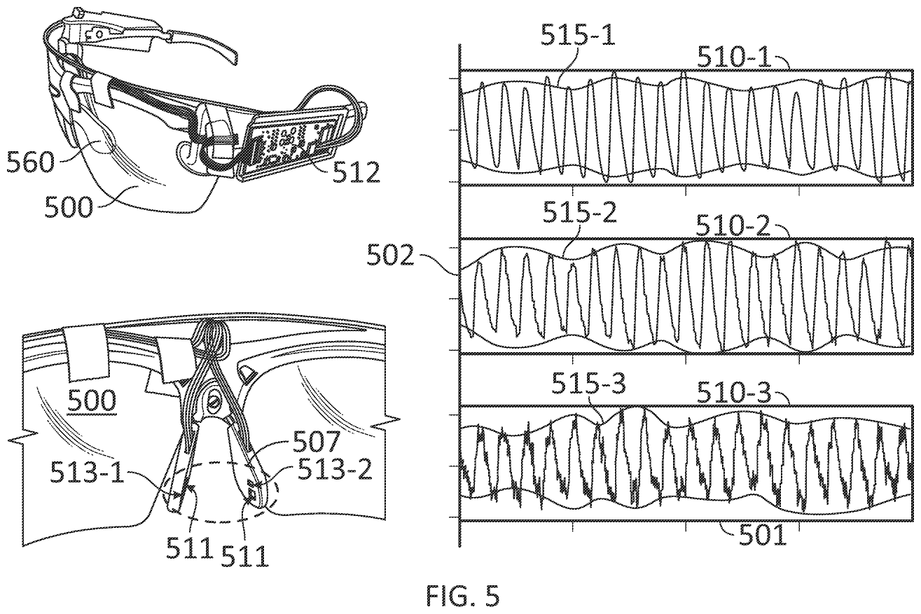

FIG. 5 illustrates waveforms 510-1, 510-2, and 510-3 (hereinafter, collectively referred to as “waveforms 510”) from receivers 511, with emitters 513-1 and 513-2 (hereinafter, collectively referred to as “emitters 513”) in different configurations in a PPG sensor 507 mounted on a smart glass or headset 500, according to some embodiments. Waveforms 510 are plotted on abscissa 501 (e.g., time in seconds) and ordinate 502 (e.g., signal amplitude, Volts, or a fraction thereof). PPG sensor 507 is mounted in nose pads 560 of the smart glass. A processor 512 may collect and process the signal from PPG sensor 507. Waveform 510-1 is from one of receivers 511 when emitter 513-2 is activated (e.g., a green LED emitter configuration in reflection mode). Emitter 513-2 for waveform 510-1 operates at 10 mA. Waveform 510-2 are generated by one of receivers 511 when emitter 513-1 (e.g., an IR LED emitter configuration in reflection mode). Emitter 513-1 for waveform 510-2 operates at 20 mA (and at a wavelength of approximately 850 nm wavelength). Waveform 510-3 are generated by one of receivers 511 with emitter 513-1 in a transmission configuration. Emitter 513-1 operates in this case at a higher current (35 mA). In some embodiments, an active front end electronic controller may provide much higher currents for the emitter (e.g., 100 mA, or even more). Waveform 510-3 clearly shows a lower signal-to-noise ratio (SNR) than the signal in waveform 510-2, for the same wavelength. The higher SNR is observed for the green LED in reflection mode.

The distance between the peaks and throughs in waveforms 510 is indicative of a heart rate. Envelopes 515-1, 515-2 and 515-3 (hereinafter, collectively referred to as “envelopes 515”) may be indicative of lower frequency events in the cardio-respiratory system (e.g., respiration rhythm, and the like). In some embodiments, waveforms 510 may be collected simultaneously, overlapping in time, or at staggered intervals, and combined to obtain a more comprehensive information of a cardiovascular state of a user.

In some embodiments, waveforms 510 from receivers 511 in PPG sensors 507 as disclosed herein may have a lower frequency envelope indicative of a respiration cycle, as illustrated.

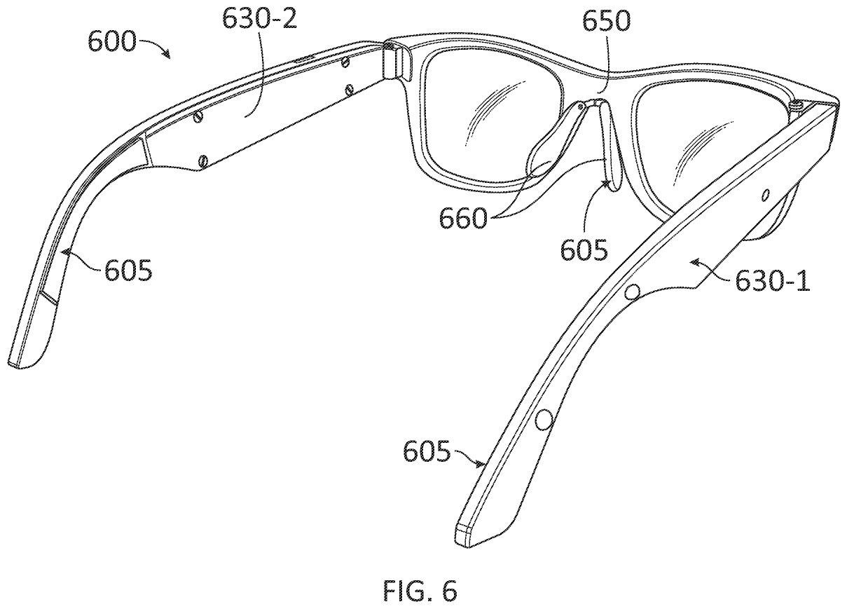

FIG. 6 illustrates different locations for electrodes 605 in an ECG or EEG sensors (e.g., EEG/ECG sensors 105) in a smart glass or headset 600, according to some embodiments. Such configurations enable to detect ECG and EEG on the user's head using custom electrodes 605 integrated into L and R temples (over/around the ears in arms 630-1 and 630-2, hereinafter “arms 630”) and on nose pads 660 mounted on a frame 650. In some embodiments, ear-to-ear measurement provides a differential voltage signal that may reduce noise and background interferences.

Nose pad electrodes 605 enable active common mode drive where an emitter in one of the nose pads interacts with a receiver in the other nose pad. Electrodes 605 may include a semi-soft electrically conductive material that enables low contact impedance. Dry metal contacts may be less desirable for user comfort. Likewise, medical-grade wet gel electrodes 605 are less desirable as users may find it uncomfortable and they may dry up through daily usage, leading to loss of contact and malfunction. In addition, nose pads and earpieces in the smart glasses may be custom-designed to improve fit and user comfort of the electrodes.

In addition to EEG and ECG signals, electrodes 605 may provide electrical waveforms indicative of motion of the eyes (Electrooculography, EOG). EOG signals from electrodes 605 in a smart glass as disclosed here may take advantage of interference removal from correlating a left-temple/right-temple signal. Other electrical signals associated with neuromuscular activity (electromyography, EMG) may be obtained from electrodes 605 disposed closer to the tip of arms 630 in smart glass 600.

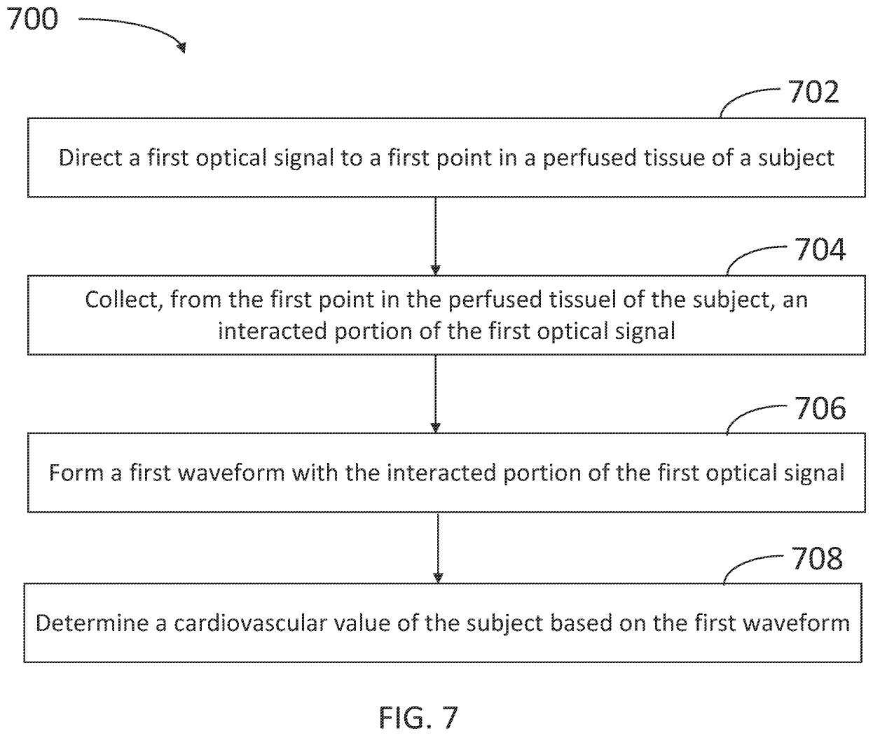

FIG. 7 is a flowchart illustrating steps in a method 700 for determining a cardiovascular condition of an AR headset user with a PPG sensor, an ECG sensor, an EEG sensor, an EOG sensor or any combination thereof (cf. PPG sensor 107, and EEG/ECG and EOG sensors 105), according to some embodiments. In some embodiments, at least one or more of the steps in method 700 may be performed by a processor executing instructions stored in a memory in either one of a smart glass or other wearable device on a user's body part (e.g., head, arm, wrist, leg, ankle, finger, toe, knee, shoulder, chest, back, and the like). In some embodiments, at least one or more of the steps in method 700 may be performed by a processor executing instructions stored in a memory, wherein either the processor or the memory, or both, are part of a mobile device for the user, a remote server or a database, communicatively coupled with each other via a network. Moreover, the mobile device, the smart glass, and the wearable devices may be communicatively coupled with each other via a wireless communication system and protocol (e.g., radio, Wi-Fi, Bluetooth, near-field communication—NFC- and the like). In some embodiments, a method consistent with the present disclosure may include one or more steps from method 700 performed in any order, simultaneously, quasi-simultaneously, or overlapping in time.

Step 702 includes directing a first optical signal to a first point in a perfused tissue of a subject. In some embodiments, step 702 includes directing a second optical signal to a second point in the perfused tissue of the subject at a known distance from the first point. In some embodiments, the first point may include a first blood vessel, and the second point may include a second blood vessel.

Step 704 includes collecting, from the first point in the perfused tissue of the subject, an interacted portion of the first optical signal. In some embodiments, step 704 includes comparing the interacted portion of the first optical signal at a first wavelength to the interacted portion of a second optical signal at a second wavelength to identify a blood oxygenation level for the subject.

Step 706 includes forming a first waveform with the interacted portion of the first optical signal. In some embodiments, step 706 includes forming a second waveform with an interacted portion of the second optical signal.

Step 708 includes determining a cardiovascular value of the subject based on the first waveform. In some embodiments, step 708 includes determining a heart rate for the subject based on a frequency of peaks in the first waveform. In some embodiments, step 708 includes identifying a blood pressure value for the subject based on a delay between the first waveform and the second waveform. In some embodiments, step 708 includes determining a cardiovascular value of the subject and includes determining a blood oxygenation level based on a differential absorption of a signal at two different wavelengths.

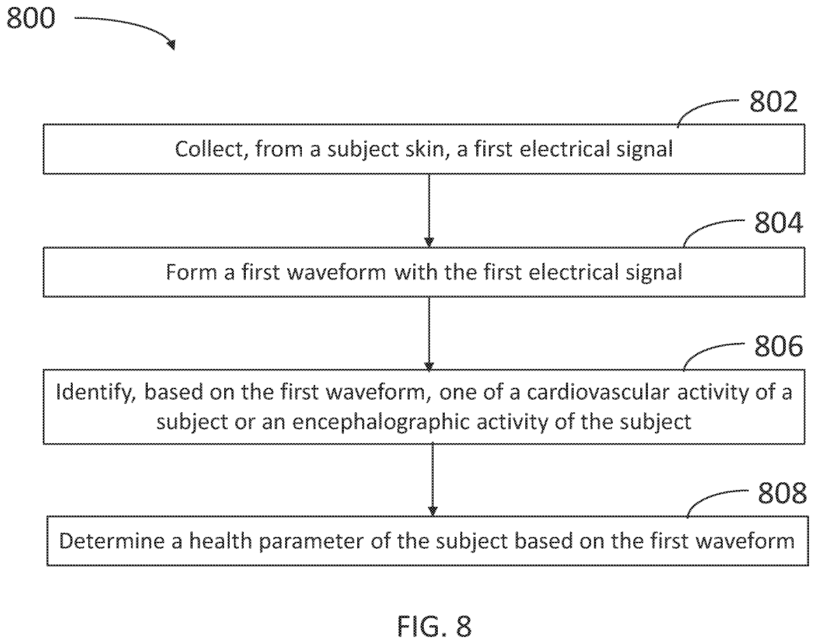

FIG. 8 is a flowchart illustrating steps in a method 800 for determining a cardiovascular condition of an AR headset user with an ECG and an EEG sensor, according to some embodiments. In some embodiments, at least one or more of the steps in method 800 may be performed by a processor executing instructions stored in a memory in either one of a smart glass or other wearable devices on a user's body part (e.g., head, arm, wrist, leg, ankle, finger, toe, knee, shoulder, chest, back, and the like). In some embodiments, at least one or more of the steps in method 800 may be performed by a processor executing instructions stored in a memory, wherein either the processor or the memory, or both, are part of a mobile device for the user, a remote server or a database, communicatively coupled with each other via a network. Moreover, the mobile device, the smart glass, and the wearable devices may be communicatively coupled with each other via a wireless communication system and protocol (e.g., radio, Wi-Fi, Bluetooth, near-field communication—NFC—and the like), or by an electrical wire. In some embodiments, a method consistent with the present disclosure may include one or more steps from method 800 performed in any order, simultaneously, quasi-simultaneously, or overlapping in time.

Step 802 includes collecting, from a subject's skin, a first electrical signal. In some embodiments, step 802 includes collecting the first electrical signal from an electrode disposed on a nose pad of an augmented reality headset, the first electrical signal being indicative of a heart activity of the subject. In some embodiments, step 802 includes collecting the first electrical signal from an electrode disposed on an arm of an augmented reality headset, the first electrical signal being indicative of a brain activity of the subject. In some embodiments, step 802 includes collecting, from a blood vessel in the subject skin, an interacted portion of an optical signal and forming a second waveform with the interacted portion. In some embodiments, step 802 further includes collecting, from a wearable device in a wrist of the subject, an interacted portion of an optical signal indicative of a blood vessel flow in the wrist of the subject and forming a second waveform with the interacted portion of the optical signal.

Step 804 includes forming a first waveform with the first electrical signal.

Step 806 includes identifying, based on the first waveform, one of a cardiovascular activity of a subject or an encephalographic activity of the subject.

Step 808 includes determining a health parameter of the subject based on the first waveform. In some embodiments, step 808 includes determining a blood pressure of the subject based on a comparison between the first waveform and the second waveform. In some embodiments, step 808 includes determining at least one of a heart condition or a neurologic condition of the subject.

Hardware Overview

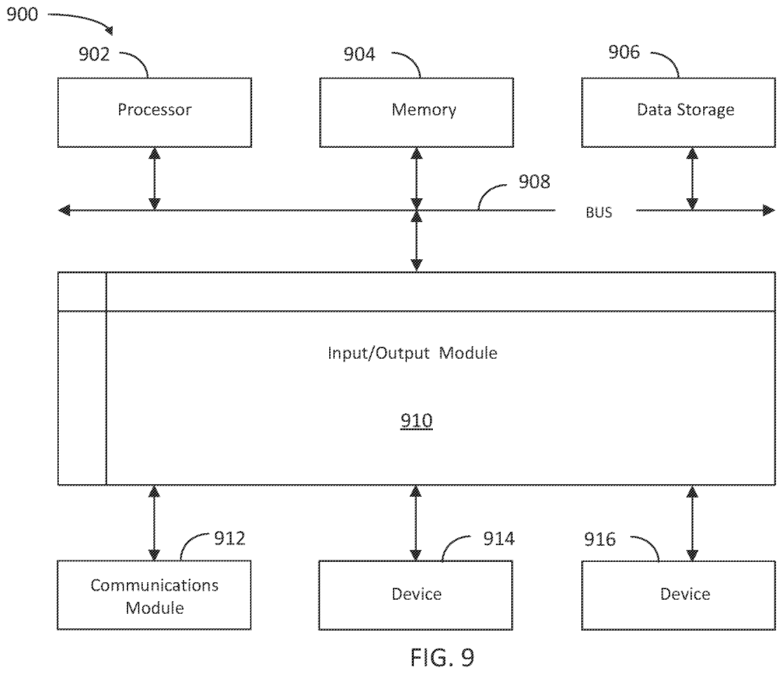

FIG. 9 is a block diagram illustrating an exemplary computer system 900 with which headsets and other client devices 110, and method 700 and 800 can be implemented, according to some embodiments. In certain aspects, computer system 900 may be implemented using hardware or a combination of software and hardware, either in a dedicated server, or integrated into another entity, or distributed across multiple entities. Computer system 900 may include a desktop computer, a laptop computer, a tablet, a phablet, a smartphone, a feature phone, a server computer, or otherwise. A server computer may be located remotely in a data center or be stored locally.

Computer system 900 includes a bus 908 or other communication mechanism for communicating information, and a processor 902 (e.g., processor 112) coupled with bus 908 for processing information. By way of example, the computer system 900 may be implemented with one or more processors 902. Processor 902 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable entity that can perform calculations or other manipulations of information.

Computer system 900 can include, in addition to hardware, code that creates an execution environment for the computer program in question, e.g., code that constitutes processor firmware, a protocol stack, a database management system, an operating system, or a combination of one or more of them stored in an included memory 904 (e.g., memory 120), such as a Random Access Memory (RAM), a flash memory, a Read-Only Memory (ROM), a Programmable Read-Only Memory (PROM), an Erasable PROM (EPROM), registers, a hard disk, a removable disk, a CD-ROM, a DVD, or any other suitable storage device, coupled with bus 908 for storing information and instructions to be executed by processor 902. The processor 902 and the memory 904 can be supplemented by, or incorporated in, special purpose logic circuitry.

The instructions may be stored in the memory 904 and implemented in one or more computer program products, e.g., one or more modules of computer program instructions encoded on a computer-readable medium for execution by, or to control the operation of, the computer system 900, and according to any method well known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multiparadigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, wirth languages, and xml-based languages. Memory 904 may also be used for storing temporary variable or other intermediate information during execution of instructions to be executed by processor 902.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors executing one or more computer programs to perform functions by operating on input data and generating output.

Computer system 900 further includes a data storage device 906 such as a magnetic disk or optical disk, coupled with bus 908 for storing information and instructions. Computer system 900 may be coupled via input/output module 910 to various devices. Input/output module 910 can be any input/output module. Exemplary input/output modules 910 include data ports such as USB ports. The input/output module 910 is configured to connect to a communications module 912. Exemplary communications modules 912 include networking interface cards, such as Ethernet cards and modems. In certain aspects, input/output module 910 is configured to connect to a plurality of devices, such as an input device 914 and/or an output device 916. Exemplary input devices 914 include a keyboard and a pointing device, e.g., a mouse or a trackball, by which a consumer can provide input to the computer system 900. Other kinds of input devices 914 can be used to provide for interaction with a consumer as well, such as a tactile input device, visual input device, audio input device, or brain-computer interface device. For example, feedback provided to the consumer can be any form of sensory feedback, e.g., visual feedback, auditory feedback, or tactile feedback; and input from the consumer can be received in any form, including acoustic, speech, tactile, or brain wave input. Exemplary output devices 916 include display devices, such as an LCD (liquid crystal display) monitor, for displaying information to the consumer.

According to one aspect of the present disclosure, headsets and client devices 110 can be implemented, at least partially, using a computer system 900 in response to processor 902 executing one or more sequences of one or more instructions contained in memory 904. Such instructions may be read into memory 904 from another machine-readable medium, such as data storage device 906. Execution of the sequences of instructions contained in main memory 904 causes processor 902 to perform the process steps described herein. One or more processors in a multi-processing arrangement may also be employed to execute the sequences of instructions contained in memory 904. In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component, e.g., a data server, or that includes a middleware component, e.g., an application server, or that includes a front end component, e.g., a client computer having a graphical consumer interface or a Web browser through which a consumer can interact with an implementation of the subject matter described in this specification, or any combination of one or more such back end, middleware, or front end components. The components of the system can be interconnected by any form or medium of digital data communication, e.g., a communication network. The communication network can include, for example, any one or more of a LAN, a WAN, a BAN, the Internet, and the like. Further, the communication network can include, but is not limited to, for example, any one or more of the following network topologies, including a bus network, a star network, a ring network, a mesh network, a star-bus network, tree or hierarchical network, or the like. The communications modules can be, for example, modems or Ethernet cards.

Computer system 900 can include clients and servers. A client and server are generally remote from each other and typically interact through a communication network. The relationship of client and server arises by virtue of computer programs running on the respective computers and having a client-server relationship to each other. Computer system 900 can be, for example, and without limitation, a desktop computer, laptop computer, or tablet computer. Computer system 900 can also be embedded in another device, for example, and without limitation, a mobile telephone, a PDA, a mobile audio player, a Global Positioning System (GPS) receiver, a video game console, and/or a television set top box.

The term “machine-readable storage medium” or “computer-readable medium” as used herein refers to any medium or media that participates in providing instructions to processor 902 for execution. Such a medium may take many forms, including, but not limited to, non-volatile media, volatile media, and transmission media. Non-volatile media include, for example, optical or magnetic disks, such as data storage device 906. Volatile media include dynamic memory, such as memory 904. Transmission media include coaxial cables, copper wire, and fiber optics, including the wires forming bus 908. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter affecting a machine-readable propagated signal, or a combination of one or more of them.

As used herein, the phrase “at least one of” preceding a series of items, with the terms “and” or “or” to separate any of the items, modifies the list as a whole, rather than each member of the list (e.g., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The word “exemplary” is used herein to mean “serving as an example, instance, or illustration.” Any embodiment described herein as “exemplary” is not necessarily to be construed as preferred or advantageous over other embodiments. Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. Relational terms such as first and second and the like may be used to distinguish one entity or action from another without necessarily requiring or implying any actual such relationship or order between such entities or actions. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public, regardless of whether such disclosure is explicitly recited in the above description. No claim element is to be construed under the provisions of 35 U.S.C. § 112, sixth paragraph, unless the element is expressly recited using the phrase “means for” or, in the case of a method claim, the element is recited using the phrase “step for.”

While this specification contains many specifics, these should not be construed as limitations on the scope of what may be described, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially described as such, one or more features from a described combination can in some cases be excised from the combination, and the described combination may be directed to a subcombination or variation of a subcombination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products.

The title, background, brief description of the drawings, abstract, and drawings are hereby incorporated into the disclosure and are provided as illustrative examples of the disclosure, not as restrictive descriptions. It is submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the described subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately described subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirements of the applicable patent law, nor should they be interpreted in such a way.

What is claimed is:

1. A headset, comprising:
a frame for holding two eyepieces, the frame having two nose pads to rest on a user's nose, and two arms to rest on two user's ears;
a sensor mounted on at least one of the nose pads or the arms, and configured to collect an optical signal from a user's perfused tissue; and
a processor configured to obtain a waveform from the optical signal and to determine a cardiovascular parameter based on the waveform.

2. The headset of claim 1, wherein the sensor includes an emitter configured to send a light to the user's perfused tissue and a receiver configured to collect at least one of a transmitted light from the user's perfused tissue, and a reflected light from the user's perfused tissue.

3. The headset of claim 1, wherein the sensor includes an emitter configured to provide a first light to the user's perfused tissue and a receiver configured to collect a second light from the user's perfused tissue, wherein the second light includes a portion of the first light, and the processor is configured to assess a magnitude for the portion of the first light.

4. The headset of claim 1, wherein the sensor is mounted on the nose pads, and the sensor includes a first emitter and a first receiver on a first nose pad and a second emitter and a second receiver on a second nose pad, wherein the first receiver is configured to receive, through the user's nose, a second light provided by the second emitter, and the second receiver is configured to receive, through the user's nose, a first light provided by the first emitter.

5. The headset of claim 1, wherein the optical signal includes at least one of a visible portion and an infrared portion of an electromagnetic spectrum.

6. The headset of claim 1, wherein multiple sensors are disposed in the nose pads and at least one of the arms, and wherein the processor is configured to identify at least one of the sensors that produces a reliable signal.

7. The headset of claim 1, wherein the sensor includes a single emitter to provide the optical signal to the user's perfused tissue and multiple receivers to collect at least a portion of the optical signal from the user's perfused tissue.

8. The headset of claim 1, wherein the sensor includes an emitter to provide the optical signal to the user's perfused tissue and at least one receiver on either side of the emitter to collect a reflected portion of the optical signal from the user's perfused tissue.

9. The headset of claim 1, wherein the sensor includes an emitter that directs the optical signal to at least one of a blood vessel adjacent to a user's skull, a blood vessel in a user's ear concha, and a blood vessel in an interstitial tissue between the user's skull and the user's ear concha.

10. The headset of claim 1, wherein the sensor includes an emitter that generates the optical signal in a green spectral range, and a receiver that collects a reflected portion of the optical signal from the user's perfused tissue.

11. A computer-implemented method, comprising:
directing a first optical signal to a first point in a perfused tissue of a subject;
collecting, from the first point in the perfused tissue of the subject, an interacted portion of the first optical signal;
forming a first waveform with the interacted portion of the first optical signal, wherein the first waveform includes a sequence of values for the interacted portion of the first optical signal collected over a period of time; and
determining a cardiovascular value of the subject based on the first waveform.

12. The computer-implemented method of claim 11, wherein determining the cardiovascular value of the subject comprises determining a heart rate for the subject based on a frequency of peaks in the first waveform.

13. The computer-implemented method of claim 11, further comprising directing a second optical signal to a second blood vessel of the subject at a known distance from the first point in the perfused tissue of the subject, forming a second waveform with an interacted portion of the second optical signal, and wherein determining the cardiovascular value of the subject comprises identifying a blood pressure value for the subject based on a delay between the first waveform and the second waveform.

14. The computer-implemented method of claim 11, further comprising comparing the interacted portion of the first optical signal at a first wavelength to the interacted portion of a second optical signal at a second wavelength to identify a blood oxygenation level for the subject.

15. A computer-implemented method, comprising:
collecting, from a subject skin, a first electrical signal;
forming a first waveform with the first electrical signal, wherein the first waveform includes a sequence of values for the first electrical signal over a period of time;
identifying, based on the first waveform, one of a cardiovascular activity of a subject or an encephalographic activity of the subject; and
determining a health parameter of the subject based on the first waveform.

16. The computer-implemented method of claim 15, wherein collecting a first electrical signal from the subject skin comprises collecting the first electrical signal from an electrode disposed on a nose pad of an augmented reality headset, the first electrical signal being indicative of a heart activity of the subject.

17. The computer-implemented method of claim 15, wherein collecting a first electrical signal from the subject skin comprises collecting the first electrical signal from an electrode disposed on an arm of an augmented reality headset, the first electrical signal being indicative of a brain activity of the subject.

18. The computer-implemented method of claim 15, further comprising collecting, from a blood vessel in the subject skin, an interacted portion of an optical signal and forming a second waveform with the interacted portion, and wherein determining a health parameter comprises determining a blood pressure of the subject based on a comparison between the first waveform and the second waveform.

19. The computer-implemented method of claim 15, further comprising collecting, from a wearable device in a wrist of the subject, an interacted portion of an optical signal indicative of a blood vessel flow in the wrist of the subject and forming a second waveform with the interacted portion of the optical signal, and wherein determining a health parameter comprises determining a blood pressure of the subject based on a comparison between the first waveform and the second waveform.

20. The computer-implemented method of claim 15, wherein determining a health parameter of the subject includes determining at least one of a heart condition or a neurologic condition of the subject.
